(19) United States
(12) Patent Application Publication
(10) Pub. No.: US 2011/0251570 A1
(43) Pub. Date: Oct. 13, 2011

(54) Disposable Covering Device and Method

(76) Inventors: Patricia Horowitz, Baltimore, MD (US); John Yacoub, Lutherville, MD (US)

(21) Appl. No.: 13/168,694

(22) Filed: Jun. 24, 2011

Related U.S. Application Data
(63) Continuation of application No. 11/831,450, filed on Jul. 31, 2007, now abandoned.

(51) Int. Cl. A61F 5/44; A61M 1/00

(52) U.S. Cl. 604/318; 604/328; 604/322

(57) ABSTRACT

A covering device is disclosed for protecting components of an indwelling catheter assembly from contamination during surgery and other medical procedures so that the assembly can be left in place during recovery. The device includes one or more covering portions that extend around the collection bag and other components of the indwelling catheter assembly to form a barrier that protects the components from contamination during the procedure. The device further includes a removal mechanism that allows the covering portions to be separated from the catheter assembly following the procedure so that the catheter assembly can be left in place during recovery, as opposed to being replaced or cleaned. In one embodiment, the covering device is provided as an integral unit with an indwelling catheter assembly. In another embodiment, the connecting device is provided separately from the indwelling catheter assembly and installed around one or more portions of the assembly prior to the procedure.

### CROSS-RELATED APPLICATION

This application is a continuation of U.S. application Ser. No. 11/831,450, filed Jul. 31, 2007. The disclosure of the above noted application is hereby incorporated by reference in its entirety.

### BACKGROUND OF THE INVENTION

1. Field of the Invention

The present invention relates generally to devices for draining fluids from body cavities during and after surgery and other medical procedures. More specifically, the invention relates to indwelling urinary catheters, such as Foley catheters and suprapubic catheters, that are inserted into body cavities as part of a medical procedure and left in place following the medical procedure. More particularly, the present invention relates to improved indwelling catheter systems that include protective covering devices that protect exposed components of the indwelling catheter systems from contamination during a medical procedure so that the systems can be left in place during recovery or ongoing treatment with reduced risk of cross-contamination.

2. Description of the Related Art

Catheters are inserted into the bladder of a patient to drain urine in connection with various medical situations. The technique, known as urinary catheterization, is used in situations in which the patient is unable to empty their own bladder. The technique is also used when a distended bladder would obstruct a practitioner's view of other organs during a medical procedure. Urinary catheterization is also performed when necessary to monitor the urine output of critically ill patients. Catheters inserted into and allowed to remain in a body cavity, such as the bladder, are often referred to as indwelling catheters. Examples of indwelling urinary catheters are the Foley catheter, which is inserted into the bladder via the urethra, and the subrapubic catheter, which is inserted into the bladder through an incision made in the abdominal wall. Indwelling catheters frequently have a balloon at one end which can be inflated with saline solution following insertion to anchor the catheter in place—i.e., to prevent the catheter from becoming dislodged from the body cavity.

Fluid drained from the body cavity is collected in a collection bag that is connected to the catheter by a connecting tube. To ensure sterility prior to insertion, the catheter, the connecting tube, and the collection bag are typically connected together and provided to practioners as an assembly within a sealed package that is opened immediately prior to use. The package may also contain a small plastic sheath that is slipped over the tip of the catheter prior to packaging. In use, the catheter assembly is removed from the package; and, if the tip of the catheter is covered by a sheath, the sheath is slipped off to expose the tip of the catheter prior to insertion. The insertion site is prepared for catheterization with an antiseptic, and the tip of the catheter is then inserted into the body until the tip of the catheter is disposed within a body cavity containing a fluid to be drained. In the case of a Foley catheter, the insertion site is the urethra and the body cavity is the bladder. When the tip of the catheter is positioned within the cavity, a balloon near the tip of the catheter is inflated using saline solution to anchor the tip within the body cavity thereby preventing the catheter from becoming dislodged from the patient. The collection bag is normally placed below the patient so that urine will drain from the catheter, through the connecting tube, and into the collection bag.

Because the connecting tube and collection bag are placed below the patient, or even on the floor during the procedure, they generally become soiled with blood, urine, feces and other contaminants. Once the catheter has been inserted, the assembly will remain in place during the procedure, and often for some period of time following the procedure. For example, the catheter assembly may be left in place during the period of time in which a patient is in the post anesthesia care unit (PACU). Because other patients are also present in the PACU, it is important that the portions of the systems which are exposed, namely the connecting tube and collection bag, be free of blood and other potentially dangerous contaminants so as to reduce the risk of cross contamination. In cases where the patient will remain in the hospital for some period of time, the catheter is sometimes left in place, which provides other opportunities for cross contamination to the patient's visitors, hospital personnel, and other patients.

Changing the connecting tube and collection bag following the procedure is not a practical solution to this cross contamination problem, because this would require opening the closed system and thus increase the risk of infection to the patient. Furthermore, it would require the use of a new connecting tube and collection bag which would increase expense. Typically, nurses or other personnel must clean the bag prior to moving the patient from the operating room to the PACU. This is time consuming and labor intensive and not an ideal solution for preventing potential cross-contamination.

Thus, there exists a need for an improved indwelling catheter system that can be left in place following a medical procedure without the need for replacement or cleaning.

### SUMMARY OF THE INVENTION

The present invention overcomes deficiencies in the prior art by providing a liquid impermeable barrier around at least one of the connecting tube and collection bag that remains in place during a medical procedure to protect the catheter assembly from contamination and is easily removed following the procedure to allow handling of the catheter assembly by medical personnel, patients and others without fear of cross-contamination.

In accordance with a first aspect of the present invention, an indwelling catheter system is provided for draining fluid from a body cavity during and after a medical procedure. The catheter system includes a catheter assembly having an indwelling catheter for insertion into a body cavity containing a body fluid and a collection bag in fluid communication with the indwelling catheter to receive body fluid drained from the body cavity via the indwelling catheter. The catheter system further includes a covering device including a first cover portion extending around the collection bag to form a barrier that protects the collection bag from contamination during a medical procedure. If the catheter assembly also includes a connecting tube extending from the indwelling catheter to the collection bag, the covering device preferably includes a second cover portion extending from the first cover portion along the connecting tube to form a barrier that also protects the connecting tube from contamination during a medical procedure. A removal mechanism integral with one or both of the first and second cover portions is manually operable to separate the first and second cover portions from the catheter assembly following the medical procedure so that the catheter assembly can be left in place without removal or cleaning.

In accordance with a second aspect of the present invention, a removable covering device for protecting exposed portions of an indwelling urinary catheter assembly from contamination during a medical procedure includes a collection bag cover including a sheet of liquid impermeable material folded to define first and second panels with free edges, the first and second panels being configured to wrap around a collection bag of an indwelling urinary catheter assembly such that the free edges are disposed opposite one another; a seal mechanism for joining the free edges to form a barrier that protects the collection bag from contamination during the medical procedure; and a removal mechanism integral with the collection bag cover to facilitate separation of the cover from the collection bag following the procedure. The covering device preferably also includes a connecting tube cover of generally tubular configuration with a slit extending lengthwise to define opposed edges, wherein the connecting tube cover is sufficiently flexible to allow opposed edges to be separated to receive a connecting tube of the indwelling catheter assembly.

In accordance with a third aspect of the present invention, a method of draining body fluids from a patient during and after a medical procedure includes the steps of inserting an indwelling catheter into a body cavity of the patient to drain fluid from the body cavity; positioning a collection bag to collect fluid drained from the body using the indwelling catheter; performing a medical procedure; protecting the collection bag from contamination with a covering device while performing the medical procedure; removing the covering device following the medical procedure; and leaving the indwelling catheter and the collection bag in place during patient recovery. The method preferably also includes the step of connecting the indwelling catheter with the collection bag using a connecting tube, wherein the protecting step includes covering the connecting tube with the covering device to protect the tube from contamination during the medical procedure.

Further applications and advantages of various embodiments of the present invention are discussed below with reference to the following drawing figures.

### BRIEF DESCRIPTION OF THE DRAWINGS

FIG. 1 is a perspective view of a Foley catheter system with a covering device according to an embodiment of the present invention.

FIG. 2 is a perspective view of a covering device for a Foley catheter assembly according to an embodiment of the present invention.

### DETAILED DESCRIPTION OF THE PREFERRED EMBODIMENTS

While the present invention may be embodied in many different forms, a number of illustrative embodiments are described herein with the understanding that the present disclosure is to be considered as providing examples of the principles of the invention and that such examples are not intended to limit the invention to preferred embodiments described and/or illustrated herein.

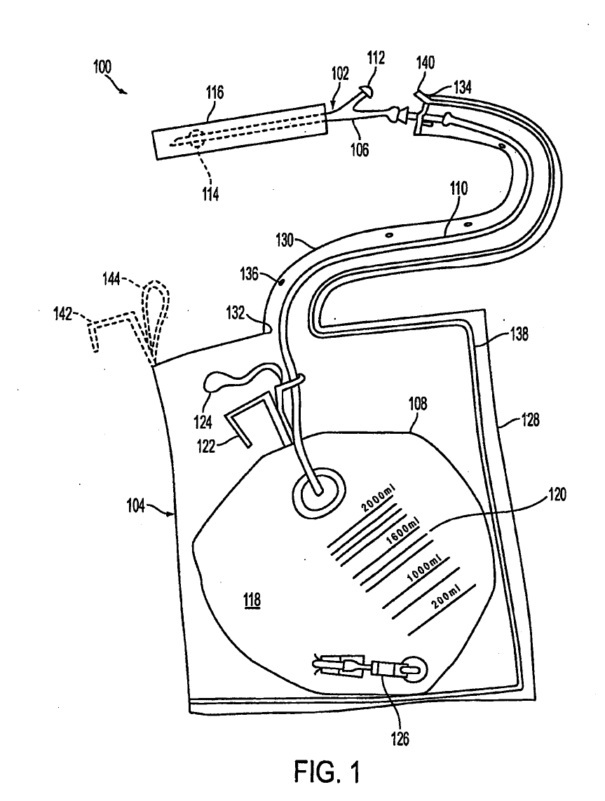

FIG. 1 is a perspective view of a Foley-type indwelling catheter system according to an embodiment of the present invention. The system includes a Foley catheter assembly and a protective covering device disposed around strategic portions of the catheter assembly to protect the covered portions from contamination during a medical procedure. The Foley catheter assembly includes a Foley catheter having a distal end or tip for insertion into a body cavity of a patient to drain bodily fluids, a collection bag for collecting bodily fluids drained from the patient, and a connecting tube extending from the collection bag to a proximal end of the catheter to provide fluid communication between the components. Catheter, collection bag, connecting tube, and sheath as further described below, constitute a Foley catheter assembly of conventional design that is well known to those of ordinary skill in the art.

Catheter is a slender tube of sufficient length to allow the proximal end of the catheter to remain outside the body when the tip of the catheter is positioned in a body cavity. An inlet port at the proximal end of the catheter permits medical personnel to inflate a balloon near the distal end or tip of the catheter to anchor the tip of the catheter within a body cavity. Also shown is a plastic sheath or sleeve that is slipped over the tip of the catheter during packaging and removed prior to use.

Collection bag is shown as a flat, circular bag with a front side and a back side (not shown). At least the front side of the collection bag is formed of a transparent material with markings thereon for measuring the volume of fluid contained within the bag. One or both of an optional hook and an optional loop of string can be affixed to the assembly to allow the bag to be hanged from a bed, a stretcher or other fixture. The collection bag also includes a drain.

In accordance with the present invention, the catheter assembly is provided with a protective covering device configured to shield the collection bag and at least a portion of the connecting tube from blood, urine, feces and other contaminants during a medical procedure and to be quickly and easily removed following the procedure so that the collection bag and the connecting tube can be handled during a subsequent period of recovery or treatment with reduced risk of cross-contamination. Covering device includes a first cover portion disposed around the collection bag and a second cover portion disposed around the connecting tube. The first cover portion is shown as a generally rectangular bag or pouch completely enclosing the collection bag and including an opening for passage of the connecting tube. The second cover portion is shown as a tubular sheath extending from the opening in the first cover portion to enclose the entire length of the connecting tube. Opening is preferably smaller is size than the collection bag so that the collection bag cannot become inadvertently dislodged from the covering device during a medical procedure. To prevent the connecting tube from becoming inadvertently exposed, the terminal or distal end of the second cover portion is preferably secured to the connecting tube using an attachment such as an adhesive strip, a sonic weld, a tie, a draw-string, a hose clamp or an elastic cuff. In addition, one or more optional vent holes can be formed in the first or second cover portions and to prevent air entrapment that might interfere with operation of the covering device. In a preferred embodiment, the first and second cover portions and are formed as an integral unit to avoid large gaps between the portions that can create a pathway for contamination.

Covering device is preferably formed of a flexible plastic material that is impermeable to fluids such as blood and urine and durable enough to withstand being stepped on and kicked without tearing or bursting, particularly when holding a collection bag containing up to 2000 ml of body fluids. The covering material is also preferably transparent to allow medical personnel to monitor fluid output by viewing the markings on the collection bag.

It will be appreciated that the durable nature of the covering device could make it difficult for medical personnel to quickly remove the device from the catheter assembly following a procedure without the use of sharp tools capable of damaging the bag or the tube. There is also risk of contaminating the bag or tube when attempting to remove the device with tools. Therefore, to facilitate a quick and clean removal of the covering device without tools, a removal mechanism is preferably provided as an integral part of the covering device. In FIG. 1, the removal mechanism is shown as a tear strip extending across almost the entire length of the covering device. The tear strip includes a tab at one end that is large enough to be grasped by medical personnel wearing sterile gloves. The tear strip is configured so that, when pulled, an opening at least as large as the collecting bag is created in the covering device to allow the collection bag and the connecting tube to be removed with minimal obstruction. To this end, the tear strip can be made of a separate strip of material that is affixed to the covering device or made by forming parallel lines of weakening (e.g., score lines) in the covering device itself.

The covering device is preferably flexible enough or otherwise configured to allow use of the hanger or loop. The covering device can optionally include an external hanger or a loop to allow the indwelling catheter system to be hanged from a bed, a stretcher, an I.V. pole, a wheelchair, or any other type of fixture. A flexible part of the covering device can incorporate such a hanger.

In use, the indwelling catheter system is preferably provided in a sterile package which is opened immediately prior to use in a medical procedure in which catheterization is indicated. The catheter system is removed from the package in a sterile environment, and the point of insertion on the patient is prepped with an antiseptic. The tip of the catheter is then inserted into the patient and advanced into a body cavity containing a fluid to be drained. Saline solution is injected into the port at the proximal end of the catheter to cause the balloon near the tip of the catheter to inflate, thereby anchoring the catheter in place during and after the procedure. Collection bag is positioned below the patient to promote gravity drainage, either by attaching the bag to a fixture in the operating room using hook or loop (alternatively, hook or loop) or by being laid on the floor where it can be kicked out of the way if necessary.

Covering device forms a liquid impermeable barrier around selected portions of the catheter assembly to protect the assembly from contaminants such as blood, urine, and feces that are inevitably found on the floor, fixtures and clothing of persons involved in the procedure. The configuration of covering device ensures that selected portions of the catheter assembly, such as the collection bag and connecting tube, do not become dislodged from the protective barrier formed by the covering device as a result of routine handling during the procedure. In addition, medical personnel can monitor fluid output from the patient by viewing the markings on the collection bag through the transparent material of the covering device.

Following the procedure, medical personnel use the removal mechanism to create an opening in the covering device large enough to remove the soiled device from the clean collection bag and the connecting tube with minimal chance of inadvertently contaminating the collection bag and connecting tube. In the case of a removal mechanism in the form of a tear strip, the medical personnel grasp the tab and pull the strip to tear a large opening in the covering device. The soiled covering device is then thrown away, while the catheter assembly is left in place to continue collecting fluid drained from the patient during recovery. Since the collection bag and connecting tube are shielded from contamination during the procedure, they can be safely handled by medical personnel, the patient and other persons without the need for replacement or cleaning once the covering device is removed.

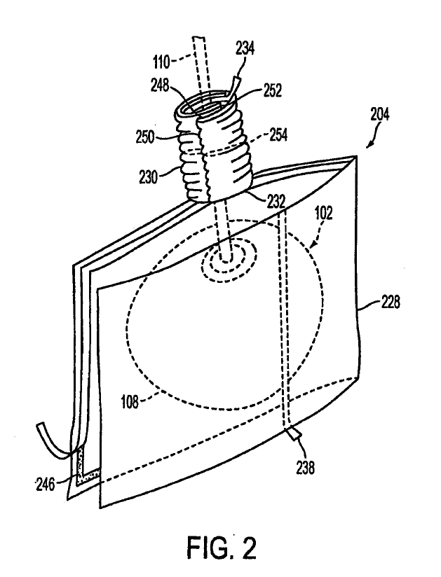

FIG. 2 shows a perspective view of a covering device for protecting a catheter assembly according to a second embodiment of the invention. The covering device can be formed of essentially the same materials and perform essentially the same functions as the device shown in FIG. 1 but is provided separate from the catheter assembly and is configured for quick attachment to, and removal from, the assembly. More specifically, the first cover portion of covering device is configured as a flat sheet large enough to be folded around the collection bag of the catheter assembly. A sealing strip, shown as an adhesive strip, is disposed along peripheral edges of the first cover portion to allow the edges of the sheet to be joined together to form a continuous seam about the collection bag with a small opening or gap for passage of the connecting tube.

The second cover portion of the covering device is configured for quick attachment to, and removal from, the connecting tube of a catheter assembly. More specifically, the second cover portion includes a generally tubular structure with a longitudinal slot having open ends. Lateral edges and of the slot overlap and are held in close proximity to one another by a plurality of elastic spring members or ribs of generally spiral configuration spaced at longitudinal intervals along the length of the cover portion. The ribs are configured such that the slot can be widened with a small amount of force to accept a connecting tube. To avoid having to work the entire length of the tube through the slot, the cover portion can be provided in a compressed or collapsed condition in which the length of the slot is effectively reduced to only a fraction of the length of the connecting tube. Thus, only a small portion of the tube need be worked through the compressed slot. Second cover portion is expandable to cover the entire length of a connecting tube, and can be provided with an attachment at a distal end to secure the second cover portion to the connecting tube in the expanded condition.

The first and second cover portions and of the covering device are preferably formed as an integral unit, with the slotted, tubular structure of the second cover portion extending from the gap in the sealing strip. A removal mechanism, similar to the tear strip shown in FIG. 1, is preferably provided along the first cover portion to facilitate removal of the covering device following a medical procedure.

In use, covering device is supplied separate from the catheter assembly and installed thereon prior to catheterization. In one embodiment, the covering device is provided in a sterile package separate from the package in which the catheter assembly is provided. Alternatively, the covering device can be provided within the same package as the catheter assembly. In either case the covering device is removed from the package, installed around the assembly, kept in place for the duration of the procedure, and removed once the procedure is completed. To install the covering device, the second cover portion is snapped onto the connecting tube in a collapsed condition (as shown) and adjusted so that the collection bag lies on one half of the first cover portion. The first cover portion is then folded around the collection bag and edges of the first cover portion joined together using the sealing strip. Once the first cover portion is sealed around the collection bag, the second cover portion is extended lengthwise along the connecting tube and secured in place using the attachment. The catheter assembly may then be used in the same manner as catheter system, with ribs holding overlapping edges of the slot in close proximity to one another to inhibit contamination of the connecting tube during the procedure.

Following the procedure, removal mechanism is used to form a large opening in the first cover portion through which the collection bag may be removed. Unlike the catheter system, however, the connecting tube may be released from the second cover portion using the slot, such that a separate removal mechanism is not needed. With the covering device removed, the catheter assembly may be left in place without the need for replacement or cleaning.

Inasmuch as the present invention is subject to many variations, modifications, and changes in detail, it is intended that all subject matter discussed above or shown in the accompanying drawings be interpreted as illustrative only and not be taken in a limiting sense. For example, while a Foley catheter assembly is used to illustrate the invention, it will be appreciated that the invention is applicable to any indwelling catheter assembly exposed to contaminants during a medical procedure and left in place with the patient for a period of time following the procedure. The shape and size of the covering device can be adjusted to accommodate any indwelling catheter assembly. The covering device can have a shape that conforms to the shape of the catheter assembly or it can have a shape that differs from the shape of the catheter assembly, so long as the covering device is sufficiently large to cover and protect portions of the catheter assembly that reside outside the body during catheterization, such as the collection bag and connecting tube. For example, in the case of a circular collection bag, the portion of the covering device that surrounds the bag can be rectangular as shown, circular like the bag, oval, triangular or of any other shape so long as it is large enough to completely cover the collection bag. Preferably, the portion of the covering device that protects the connecting tube is narrower in width than the portion that protects the collection bag to fix the covering device in place around the catheter assembly. The portion protecting the collection bag can be flat or provided with gussets or folds to allow for expansion of the collection bag as it fills with body fluids. Furthermore, while the covering device is described as having first and second portions formed as an integral unit, it will be appreciated that the first and second portions can be provided as separate units to protect different parts of the catheter assembly. While the covering device is described as being made of a transparent plastic material, it could be made of an opaque or translucent material with an opening or window formed therein in registration with markings on the collection bag to allow medical personnel to monitor the fluid output from the patient. Alternatively, the back of the covering device can be opaque while the front is clear. The covering device can be made of a single layer of material or of multiple layers to enhance the strength of the device or adjust the amount of protection being provided. The covering device can have flaps or openings to allow the collection bag to be drained without removing the covering device or to allow the hanging hook of the collection bag to be accessed and utilized. The hook can be disposed in the first portion of the covering device with the collection bag, or in the second portion of the covering device with the connecting tube. The covering device can be configured to protect either or both of the connecting tube and the collection bag. Similarly, the covering device can be configured to cover the entire length of the connecting tube or only a portion of the connecting tube, depending upon the application. The removal mechanism can include tear strips as shown, seams closed by fasteners (e.g., hook and loop fasteners, Ziploc-type closures, drawstrings, self-sealing adhesives or cling-type materials, snaps, etc.), lines of perforation (which can also function as vents to prevent air entrapment), and other mechanisms integrated into the covering device to facilitate the creation of openings allowing the covering device to be quickly and easily removed from the catheter assembly.

1. An indwelling catheter system for draining fluid from a body cavity during and after a medical procedure, said catheter system comprising: a catheter assembly including an indwelling catheter for insertion into a body cavity containing a body fluid and a collection bag in fluid communication with said indwelling catheter to receive body fluid drained from the body cavity via said indwelling catheter; and a covering device including a first cover portion extending around said collection bag to form a barrier that protects said collection bag from contamination during a medical procedure and a first removal mechanism integral with said first cover portion and manually operable to separate said first cover portion from said collection bag following the medical procedure.

2. The indwelling catheter system of claim 1, wherein said catheter assembly further includes a connecting tube extending from said indwelling catheter to said collection bag, and wherein said covering device includes a second cover portion extending from said first cover portion along said connecting tube to form a barrier that protects said connecting tube from contamination during a medical procedure and a second removal mechanism integral with said second cover portion and manually operable to separate said second cover portion from said connecting tube following the medical procedure.

3. The indwelling catheter system of claim 2, wherein said first and second cover portions are formed as an integral unit.

4. The indwelling catheter system of claim 2, wherein said catheter assembly includes a coupling between said indwelling catheter and said connecting tube, and wherein said second cover portion extends from said first cover portion to said coupling.

5. The indwelling catheter system of claim 1, wherein said first removal mechanism includes a tear strip extending across at least a portion of said first cover portion.

6. The indwelling catheter system of claim 2, wherein said second removal system includes a tear strip extending across at least a portion of said second cover portion.

7. The indwelling catheter system of claim 3, wherein said first and second removal systems include a single tear strip extending across said first and second cover portions.

8. The indwelling catheter system of claim 1, wherein said first removal mechanism includes a line of weakening extending across at least a portion of said first cover portion.

9. The indwelling catheter system of claim 2, wherein said second removal system includes a line of weakening extending across at least a portion of said second cover portion.

10. The indwelling catheter system of claim 3, wherein said first and second removal systems include a line of weakening that extends across said first and second cover portions.

11. The indwelling catheter system of claim 2, wherein said second covering portion extends along said connecting tube from said first covering portion to a terminal end secured to said connecting tube.

12. The indwelling catheter system of claim 1, wherein said collection bag includes a fill indicator and wherein at least a portion of said first cover portion covering said fill indicator is transparent.

13. The indwelling catheter system of claim 1, wherein said catheter assembly further includes a hook associated with said collection bag, and wherein at least one of said first and second cover portions extends around said hook to allow said catheter assembly to be suspended during the medical procedure using said hook.

14. The indwelling catheter system of claim 1, further comprising a sealed package, wherein said catheter assembly is provided with said covering device in a sterile condition within said sealed package.

15. A removable covering device for protecting exposed portions of an indwelling urinary catheter assembly from contamination during a medical procedure, said covering device comprising: a collection bag cover including a sheet of liquid impermeable material folded to define first and second panels with free edges, said first and second panels being configured to wrap around a collection bag of an indwelling urinary catheter assembly such that said free edges are disposed opposite one another; a seal mechanism for joining said free edges to form a barrier that protects the collection bag from contamination during the medical procedure; and a removal mechanism integral with said collection bag cover to facilitate separation of said cover from the collection bag.

16. The removable covering device of claim 15, wherein said seal mechanism includes hook and loop fasteners.

17. The removable covering device of claim 15, wherein said seal mechanism includes at least free edges of said bag cover being formed of a self-sealing material.

18. The removable covering device of claim 15, wherein said seal mechanism includes an adhesive strip.

19. The removable covering device of claim 15, wherein said removal mechanism includes a tear strip extending across said collection bag cover.

20. The removable covering device of claim 15, wherein said removal mechanism includes a line of weakening extending across said collection bag cover.

21. The removable covering device of claim 20, wherein said line of weakening includes a plurality of perforations.

22. The removable covering device of claim 15, further comprising a connecting tube cover of generally tubular configuration with a slit extending lengthwise to define opposed edges, said connecting tube cover being sufficiently flexible to allow opposed edges to be separated to receive a connecting tube of the indwelling catheter assembly.

23. The removable covering device of claim 22, wherein opposed edges of said connecting tube cover overlap.

24. The removable covering device of claim 22, wherein said connecting tube cover is collapsible in a lengthwise direction.

25. The removable covering device of claim 24, wherein said connecting tube cover includes a terminal end disposed distally of said collection bag cover and an attachment mechanism for securing said terminal end to a collecting tube of an indwelling catheter assembly.

26. The removable covering device of claim 24, wherein said connecting tube cover includes a flexible tubular body and a plurality of spaced spring elements attached to said tubular body to maintain said edges in closely spaced relation to one another.

27. The removable covering device of claim 22, wherein said collection bag cover and said connecting tube cover are formed as an integral unit.

28. A method of draining body fluids from a patient during and after a medical procedure, said method comprising the steps of: inserting an indwelling catheter into a body cavity of the patient to drain fluid from the body cavity; positioning a collection bag to collect fluid drained from the body using the indwelling catheter; performing a medical procedure; protecting the collection bag from contamination with a cover device while performing the medical procedure; removing the cover device following the medical procedure; and leaving the indwelling catheter and the collection bag in place during patient recovery.

29. The method of claim 28, wherein the cover device includes a liquid impermeable sheet of material and said protecting step includes wrapping the sheet of material around the collection bag and sealing free edges of the sheet to protect the collection bag from contamination during the medical procedure.

30. The method of claim 28, further comprising the step of connecting the indwelling catheter with the collection bag using a connecting tube, wherein said protecting step includes covering the connecting tube with the cover device to protect the tube from contamination during the medical procedure.

31. The method of claim 30, wherein said protecting step includes positioning a portion of the cover device around the connecting tube in a collapsed condition and extending the collapsed portion of the cover device lengthwise to protect the connecting tube from contamination during the medical procedure.

32. The method of claim 28, wherein said removing step includes tearing the cover device using an integrated removal mechanism.

33. The method of claim 30, wherein the portion of the cover device protecting the connecting tube includes a slit, and wherein said removing step includes slipping the connecting tube through the slit in the cover device.
